(19) United States
(12) Patent Application Publication
(10) Pub. No.: US 2024/0116839 A1
(43) Pub. Date: Apr. 11, 2024

(54) PROCESS FOR PREPARING BISPHENOL A (BPA) IN THE PRESENCE OF ACETOPHENONE

(71) Applicant: Covestro Deutschland AG, Kaiser-Wilhelm-Allee 60 (DE)

(72) Inventors: Jerico Jayson Uy, Antwerpen (BE); Erik Sluyts, Brasschaat (BE)

(21) Appl. No.: 18/270,364

(22) PCT Filed: Feb. 16, 2022
(86) PCT No.: PCT/EP2022/053775

§ 371 Date: Jun. 29, 2023

(30) Foreign Application Priority Data
Feb. 23, 2021 (EP) 21158689.6

(51) Int. Cl. C07C 37/20; C07C 37/84; C08G 64/30

(52) U.S. Cl. CPC C07C 37/20; C07C 37/84; C08G 64/307

(57) ABSTRACT

The present invention relates to a process for preparing bisphenol A in the presence of acetophenone without poisoning the catalyst system comprising an ion exchange resin catalyst and a sulfur containing cocatalyst. Moreover, the present invention provides a process for preparing polycarbonate.

### CROSS REFERENCE TO RELATED APPLICATIONS

This application is a U.S. national stage application, filed under 35 U.S.C. § 371, of International Application No. PCT/EP2022/053775, which was filed on Feb. 16, 2022, and which claims priority to European Patent Application No. 21158689.6, which was filed on Feb. 23, 2021. The contents of which are hereby incorporated by reference into this specification.

### FIELD

The present invention relates to a process for preparing bisphenol A and a process for preparing polycarbonate.

### BACKGROUND

Bisphenol A or BPA is an important monomer in the production of polycarbonate or epoxy resins. Normally, BPA is used in the form of para,para-BPA (2,2-Bis(4-hydroxyphenol)propane; p,p-BPA). However, in the production of BPA also ortho, ortho-BPA (o,o-BPA) and/or ortho,para-BPA (o,p-BPA) may be formed. In principle, when referring to BPA, reference is made to para,para-BPA still containing low amounts of ortho, ortho-BPA and/or ortho,para-BPA.

According to the state of the art BPA is produced by reacting phenol with acetone in the presence of an acid catalyst to give the bisphenol. In former times hydrochloric acid (HCl) was used for the commercial process of the condensation reaction. Today, a heterogeneous continuous process for the production of BPA is used in the presence of an ion exchange resin catalyst, wherein said ion exchange resin comprises a crosslinked acid functionalized polystyrene resin. The most important resins are crosslinked polystyrenes with sulfonic acid groups. Divinylbenzene is mostly used as the crosslinking agent as described in GB849965, U.S. Pat. No. 4,427,793, EP0007791 and EP0621252 or Chemistry and properties of crosslinked polymers, edited by Santokh S. Labana, Academic Press, New York 1977.

In order to achieve a high selectivity, the reaction of phenol with acetone can be performed in the presence of suitable co-catalyst. US2005/0177006 A1 and U.S. Pat. No. 4,859,803 describe a process for preparing bisphenol A in the presence of an ion-exchange catalyst and mercaptopropionic acid or a mercaptan as co-catalyst. It is known that the catalyst deactivates over time. For example, the deactivation is described in EP0583712, EP10620041, DE14312038. One major objective for the production process is to maximize the performance and dwell times of the catalyst system. Accordingly, there is a need to identify potential poisonous substances, byproducts, impurities of educts etc. in order to deal with this objective.

WO2012/150560 A1 teaches the use of a specific catalyst system comprising an ion exchange resin catalyst and a sulfur containing cocatalyst, wherein the co-catalyst is chemically bound to the ion exchange resin catalyst, and also a process for catalyzing condensation reactions between phenols and ketones using such specific catalyst system. Furthermore WO2012/150560 A1 discloses a process for catalyzing condensation reactions between phenols and ketones that does not utilize a bulk promoter that is not chemically bound to the ion exchange resin catalyst.

In the same way EP1520617 A1 describes a process for preparing bisphenols in the presence of an acidic ion-exchange resin catalyst which is modified with specific cationic compound.

U.S. Pat. No. 8,247,619B2 describes the production of BPA based on bio-derived phenol and/or bio-derived acetone in the presence bio-derived impurities in the educts. This document solely describes the use of an ion exchange resin catalyst with attached promotor meaning that the co-catalyst is chemically (i. e. ionically) bound to the ion exchange resin catalyst. Catalyst poisoning has not been determined in this prior art document.

Acetophenone is one of the impurities which can be present in raw phenol. As described above, normally impurities are tried to be avoided or their amount is reduced as low as possible in order to avoid any side-reactions, poisoning of the catalyst etc. in the desired reaction.

However, the removal of acetophenone from raw phenol (either fossil based or bio-derived) consumes time and money and thus, renders the raw phenol more expensive. In the end it increases the costs of bisphenol A and the respective polymer prepared from this bisphenol A. Moreover, the concentration of acetophenone in raw phenol varies depending on the supplier and their process of purification of these raw materials. This means that different raw material qualities need to be handled (e. g. another step of purification needs to be performed if the specification exceeds a certain threshold) decreasing flexibility of the process and choice of raw material supplier.

### SUMMARY

Therefore, it was an object of the present invention to provide a process for the preparation of ortho,para-, ortho, ortho- and/or para,para-bisphenol A via condensation of phenol and acetone which is more economical than the processes of the prior art. Moreover, it was an object of the present invention to provide a process for the preparation of ortho,para-, ortho, ortho- and/or para,para-bisphenol A via condensation of phenol and acetone which is more flexible and/or which allows more flexibility in the choice of the quality of raw phenol. This flexibility should be preferably provided with respect to the concentration of acetophenone as impurity in raw phenol.

At least one of the above-mentioned objects, preferably all of these objects have been solved by the present invention. Surprisingly, it has been found that a catalyst system comprising an ion exchange resin catalyst and a sulfur containing cocatalyst is not susceptible to catalyst poisoning by acetophenone. Moreover, it has been found that a catalyst system comprising an ion exchange resin catalyst and a sulfur containing cocatalyst, wherein at least part of the sulfur containing cocatalyst is neither covalently nor ionically bound (i. e. not chemically bound) to the ion exchange resin catalyst, is not susceptible to catalyst poisoning by acetophenone. Moreover, normally the amount of acetophenone in raw phenol is as low as possible. Due to the fact that the specific catalyst system of the present invention is not affected by this impurity, cheaper raw phenol can be used without the risk of reducing catalyst life time. This renders the overall process more cost effective. In addition, as less energy for purifying the raw materials is needed, the process becomes more ecologically advantageous. Moreover, the process allows more flexibility in the choice of the quality of raw phenol, especially with respect to the concentration acetophenone in those raw materials.

Accordingly, the present invention provides a process for preparing ortho,para-, ortho, ortho- and/or para,para-bisphenol A comprising the step of
(a) condensing raw phenol and raw acetone in the presence of a catalyst system, wherein the catalyst system comprises an ion exchange resin catalyst and a sulfur containing cocatalyst,
characterized in that the amount of acetophenone present in step (a) is higher than 1 ppm with respect to the total weight of the raw phenol.

### DETAILED DESCRIPTION

According to the present invention reference is made to “raw phenol” and/or “raw acetone”. The term “raw” is used for the unreacted educts as applied, especially added, in the process for preparing BPA. In particular, this term is used to distinguish the phenol which is freshly added to the reaction (as raw phenol) and the phenol which is recycled in the process for preparing BPA (recycled phenol). Such recycled phenol cannot add additional acetophenone to the process. The same holds true for acetone which is freshly added to the reaction (as raw acetone) and acetone which is recycled in the process for preparing BPA (recycled acetone). When referring to phenol and/or acetone without any further specification it is preferred that the sum either the chemical compound as such or both raw and recycled phenol and/or raw and recycled acetone are meant.

Acetophenone is an impurity in raw phenol which is one of the educts of the reaction of BPA. Raw phenol can contain acetophenone impurities. For example, the production pathways for phenol are described in Arpe, Hans-Jürgen, Industrielle Organische Chemie, 6. Auflage, January 2007, Wiley-VCH. In particular the process for preparing phenol is described in Ullmann's Encyclopedia of Industrial Chemistry, chapters Phenol and Phenol derivatives. The oxidation of cumene, also known as Hock process, is by far the dominant synthetic route to phenol. Among the contaminants formed during the manufacture of phenol acetophenone.

The process of the present invention is characterized in that the amount of acetophenone present in step (a) is higher than 1 ppm, preferably higher than 1.5 ppm, more preferably higher than 2 ppm, still more preferably higher than 2.5 ppm, still preferably higher than 3 ppm, still more preferably higher than 4 ppm, still more preferably higher than 5 ppm, still more preferably higher than 6 ppm, still more preferably higher than 7 ppm, still more preferably higher than 8 ppm, still more preferably higher than 9 ppm, still more preferably higher than 10 ppm, still more preferably higher than 11 ppm, still more preferably higher than 12 ppm, still more preferably higher than 13 ppm, still more preferably higher than 15 ppm, still more preferably higher than 20 ppm and most preferably higher than 50 ppm with respect to the total weight the raw phenol.

Moreover, it is preferable that the amount of acetophenone present in step (a) is higher than 1 ppm and equal to or lower than 5000 ppm, more preferably equal to or lower 4500 ppm, still more preferably equal to or lower 4000 ppm, still more preferably equal to or lower 3500 ppm, still more preferably equal to or lower 3000 ppm, still more preferably equal to or lower 2500 ppm and most preferably equal to or lower 2000 ppm with respect to the total weight of the raw phenol. It is understood that the upper limits given here can be combined with the preferred lower limits given above can be combined. The skilled person knows how to determine the amount of MBF in raw phenol. For example, the amount of 2-acetophenone in raw phenol can be determined according to ASTM D6142-12 (2013).

According to the present invention “ppm” preferably refers to parts by weight.

Preferably, the process of the present invention is characterized in that the acetophenone is present throughout the whole process step (a). According to the present invention it has been found that when using the catalyst system of the present invention, the acetophenone does not seem to react during process step (a) or only reacts to a very little extend. This means that at least some, preferably all of the acetophenone is still present at the end of process step (a) and/or also in the resulting BPA. Preferably at least 25 wt.-%, more preferably at least 50 wt.-% and most preferably at least 75 wt.-% with respect to the acetophenone being present at the beginning of process step (a) are present also at the end of process step (a), preferably at the beginning of process step (b) described below. Preferably at least 25 wt.-%, more preferably at least 50 wt.-% and most preferably at least 75 wt.-% with respect to the acetophenone being present at the beginning of process step (a) are present in the resulting ortho,para-, ortho, ortho- or para,para-bisphenol A.

Preferably, the process of the present invention is characterized in that the process additionally comprises the following step:
(b) separating the mixture obtained after step (a) into a bisphenol A fraction comprising at least one of ortho,para-, ortho, ortho- or para,para-bisphenol A and a phenol fraction, wherein the phenol fraction comprises unreacted phenol and acetophenone.

Preferably, the bisphenol A fraction is taken as product and/or further purified. Several variants of production processes exist to provide the bisphenol of high purity. This high purity is especially of importance for the use of BPA as monomer in the production of polycarbonates. WO-A 0172677 describes crystals of an adduct of a bisphenol and of a phenol and a method for producing these crystals and finally preparing bisphenols. It was found that by crystallizing these adducts a para,para-BPA of high purity can be obtained. EP1944284 describes the process for producing bisphenols wherein the crystallization comprises continuous suspension crystallization devices. It is mentioned that the requirements with respect to the BPA purity are increasing and that with the disclosed method a very pure BPA of higher than 99.7% can be obtained. WO-A 2005075397 describes a process for producing bisphenol A in which the water that is produced during the reaction is removed by distillation. By this method the unreacted acetone is recovered and recycled resulting in an economically favorable process.

Preferably, the process of the present invention is characterized in that the separation in step (b) is performed using a crystallization technique. Still preferably, the separation in step (b) is performed using at least one continuous suspension crystallization device.

It has been further described to make use of a mother liquor cycle. BPA is taken out of the solvent by crystallization and filtration after the reaction. The mother liquor typically contains 5 to 20% BPA and byproducts dissolved in unreacted phenol. Moreover, water is formed during the reaction and is removed from the mother liquor in the dewatering section. Preferably, the fraction comprising unreacted phenol is recycled for further reaction. This preferably means that the mother liquor is recycled. It is re-used as unreacted phenol in the reaction with acetone in order to give BPA. The flow of mother liquor is preferably conventionally recirculated into the reaction unit.

Typically byproducts in the mother liquor are for example o,p-BPA, o,o-BPA, substituted indenes, hydroxyphenyl indanoles, hydroxyphenyl chromanes, substituted xanthenes and higher condensed compounds. In addition, further secondary compounds such as anisole, mesityl oxide, mesitylene and diaceton alcohol may be formed as a result of self-condensation of the acetone and reaction with impurities in the raw material.

Due to the recycling of mother liquor byproducts accumulate in the circulation stream and can lead to an additional deactivation of the catalyst system. This means for a prolonged use of a catalyst, the impact of initial impurities in the educts have to be considered as well as the impact of possible byproducts in the reaction itself, resulting either from the reaction of phenol with acetone or from a reaction of one of the impurities.

Still preferably, the process according to the present invention is characterized in that the process comprises the additional step of
(c) using at least a part of the phenol fraction obtained in step (b) as educt in step (a).

Still preferably, the part of the phenol fraction comprises at least 10 wt.-%, more preferably at least 25 wt.-% and most preferably at least 50 wt.-% of acetophenone, wherein the weight percent of acetophenone refers to the part of acetophenone as compared to the acetophenone being present in the raw phenol.

In order to avoid accumulation of the introduced acetophenone, byproducts and/or impurities (either added with the raw educts or potentially formed due to the presence of acetophenone in step (a) in the system) several options exist. Those options include inter alia the purge stream, the waste water, the off gas and the BPA as product itself. The major one seems to be the purge stream, for example a portion of the mother liquor is discharged. Another approach comprises the passing a part of the entire amount of the circulation stream after solid/liquid separation and before or after the removal of water and residual acetone, over e. g. a rearrangement unit filled with acid ion exchanger. In this rearrangement unit some of the byproducts from BPA preparation are isomerized to give p,p-BPA. It is assumed that the acetophenone can be at least partly removed by a purge stream. Accordingly, it is preferred that at least part of the phenol fraction obtained in step (b) is used as educt in step (a), wherein at least a part of this stream is purged. Preferably, more than 50 vol.-% of the phenol fraction obtained in step (b) is used as educt in step (a), wherein the vol.-% is based on the total volume of the phenol fraction.

According to the present invention, a catalyst system is used which comprises an ion exchange resin and a sulfur containing cocatalyst. These catalyst systems are known to the skilled person. Especially two different types of catalyst systems exist. One is mostly referred to as “promoted catalyst” and the other as “unpromoted catalyst”. The promoted catalyst comprises a cocatalyst which is attached to a portion of the ion exchange resin. This attachment is either ionic or covalent in nature. Examples for such promoted catalyst systems are for example described in WO2012/150560A1, US2004/0192975 A1, U.S. Pat. No. 8,247,619 B or U.S. Pat. No. 5,414,151 B. On the other hand, in the “unpromoted catalyst” system the cocatalyst is typically not attached to the ion exchange resin.

The ion exchange resin which can be used in the process of the present invention is known by the skilled person. Preferably, it is an acidic ion exchange resin. Such ion exchange resin can have from 2% to 20%, preferably 3 to 10% and most preferably 3.5 to 5.5% crosslinkage. The acidic ion exchange resin preferably can be selected from the group consisting of sulfonated styrene divinyl benzene resins, sulfonated styrene resins, phenol formaldehyde sulfonic acid resins and benzene formaldehyde sulfonic acid. Moreover, the ion exchange resin may contain sulfonic acid groups. The catalyst bed can be either a fixed bed or a fluidized bed.

Furthermore, the catalyst system of the present invention comprises a sulfur containing cocatalyst. The sulfur containing cocatalyst can be one substance or a mixture of at least two substances. Preferably, the sulfur containing cocatalyst is selected from the group consisting of mercaptopropionic acid, hydrogen sulfide, alkyl sulfides such as ethyl sulfide, mercaptoalkylpyridines, mercaptoalkylamines, thiazolidines, aminothiophenols and mixtures thereof. In the case of the promoted catalyst the sulfur containing cocatalyst is preferably selected from mercaptoalkylpyridines, such as 3-mercaptomethylpyridine, 3-(2-mercaptoethyl)pyridine and 4-(2-mercaptoethyl)pyridine; mercaptoalkylamines, such as 2-mercaptoethylamine, 3-mercaptopropylamine and 4-mercaptobutylamine; thiazolidines, such as thiazolidine, 2-2-dimethylthiazolidine, 2-methyl-2-phenylthiazolidine and 3-methylthiazolidine; aminothiophenols such as 4-methylthiophenol and mixtures thereof. In case of the unpromoted catalyst, the sulfur containing cocatalyst is selected from the group consisting of mercaptopropionic acid, hydrogen sulfide, alkyl sulfides such as ethyl sulfide and mixtures thereof. According to the present invention, preferably an unpromoted catalyst system is used. This means that it is preferred that in the catalyst system at least part, preferably at least 75 mol-% of the sulfur containing cocatalyst is neither covalently nor ionically bound to the ion exchange resin catalyst at the beginning of process step (a).

In this case the cocatalyst is preferably dissolved in the reaction solution of process step (a). Still preferably, the cocatalyst is dissolved homogenously in the reaction solution of process step (a). Preferably, the process of the present invention is characterized in that the sulfur containing cocatalyst is selected from the group consisting of mercaptopropionic acid, hydrogen sulfide, alkyl sulfides such as ethyl sulfide and mixtures thereof. Most preferably, the sulfur containing cocatalyst is 3-mercaptopropionic acid.

Preferably, the catalyst system of the present invention comprises a sulfur containing cocatalyst, wherein all of the sulfur containing cocatalyst is neither covalently nor ionically bound to the ion exchange resin catalyst. This means that preferably all of the sulfur containing cocatalyst is added to the process step (a). According to the present invention the expression “not chemically bound” or “neither covalently nor ionically bound” refers to a catalyst system where neither a covalent nor an ionic bound between the ion exchange resin catalyst and the sulfur containing cocatalyst is present at the beginning of process step (a). However, this does not mean that at least part of the sulfur containing cocatalyst might get fixed to the heterogeneous catalyst matrix via ionic or covalent bonds. Nevertheless, at the beginning of process step (a) no such ionic or covalent bonds of the sulfur containing cocatalyst are present, but if they are formed at all, they are formed over time. Accordingly, preferably the sulfur containing cocatalyst is added to process step (a). The term “added” describes an active process step. This means, as said above, that the cocatalyst is preferably dissolved in the reaction solution of process step (a). Additionally the cocatalyst can be added at any other process step or even twice or more times at process step (a). Moreover, preferably, most of the sulfur containing cocatalyst is neither covalently nor ionically bound to the ion exchange resin catalyst. This means that at least 75 mol-%, still preferably at least 80 mol-%, most preferably at least 90 mol-% of the sulfur containing cocatalyst is not chemically bound to the ion exchange resin catalyst. Here the mol-% relate to the total sum of the cocatalyst present in process step (a).

Because acetophenone is a common impurity in raw phenol, it is preferred that the acetophenone present in step (a) is introduced into the process step (a) as impurity in the raw phenol. Nevertheless, at least part of the acetophenone can be present in process step (a) due to other reasons. For example, some of the acetophenone present in process step (a) may be present due to the recycling of phenol.

According to the present invention, it is possible that the raw phenol and/or the raw acetone which are used in process step (a) are bio-based.

As used according to the present invention, the term “bio-derived” or “bio-based” refers to (raw) phenol and/or (raw) acetone from a currently renewable resource. In particular, this term is used as opposed to phenol being derived from fossil fuels. The fact whether a raw material is bio-based, can be verified by measurements on carbon isotope levels, since the relative amounts of isotopic carbon C14 are lower in fossil-fuel materials. The skilled person knows such measurements which can be performed for example according to ASTM D6866-18 (2018) or ISO16620-1 to -5 (2015).

In another aspect the present invention provides a process for preparing polycarbonate comprising the steps of
(i) obtaining a ortho,para-, ortho, ortho- and/or para,para-bisphenol A according to the process of the present invention in any embodiment or combination of preferred embodiments and
(ii) polymerizing the ortho,para-, ortho, ortho- and/or para,para-bisphenol A obtained in step (i), optionally in the presence of at least one further monomer in order to obtain a polycarbonate.

As explained above, the process for the production of ortho,para-, ortho, ortho- and/or para,para-bisphenol A of the present invention provides a BPA which can be obtained in a more economical and/or ecological way. Accordingly, in using this BPA as obtained with the process according to the present invention, the process for preparing polycarbonate according to the present invention is more economical and/or ecological, too.

Reaction step (ii) is known to the skilled person. The polycarbonates can be prepared in a known manner from the BPA, carbonic acid derivatives, optionally chain terminators and optionally branching agents by interphase phosgenation or melt transesterification.

In the interphase phosgenation bisphenols and optionally branching agents are dissolved in aqueous alkaline solution and reacted with a carbonate source, such as phosgene, optionally dissolved in a solvent, in a two-phase mixture comprising an aqueous alkaline solution, an organic solvent and a catalyst, preferably an amine compound. The reaction procedure can also be effected in a plurality of stages. Such processes for the preparation of polycarbonate are known in principle as interfacial processes, for example from H. Schnell, Chemistry and Physics of Polycarbonates, Polymer Reviews, Vol. 9, Interscience Publishers, New York 1964 page 33 et seq., and on Polymer Reviews, Vol. 10, “Condensation Polymers by Interfacial and Solution Methods”, Paul W. Morgan, Interscience Publishers, New York 1965, chapter VIII, page 325, and the underlying conditions are therefore familiar to the person skilled in the art.

Alternatively, polycarbonates may also be prepared by the melt transesterification process. The melt transesterification process is described, for example, in Encyclopaedia of Polymer Science, Vol. 10 (1969), Chemistry and Physics of Polycarbonates, Polymer Reviews, H. Schnell, Vol, 9, John Wiley and Sons, Inc. (1964), and DE-C 10 31 512. In the melt transesterification process, the aromatic dihydroxy compounds already described in the case of an interfacial process are transesterified with carbonic acid diesters with the aid of suitable catalysts and optionally further additives in the melt.

Preferably, the process for preparing polycarbonates according to the present invention is characterized in that the process step (i) further comprises a step of purifying the ortho,para-, ortho, ortho- and/or para,para-bisphenol A in order to reduce the amount of acetophenone. As has been described above, cheaper raw phenol can be used in the process of the present invention. However, when having acetophenone as impurity in these cheaper raw materials, this impurity does not seem to substantially react. Therefore, the acetophenone is preferably removed before the polymerization. This removal is preferably conducted using the above-described crystallization technique.

### Examples

Materials used in the examples:

| | |
|---|---|
| catalyst | acidic polymer-based resin (polystyrene-divinyl- |
| | benzene) with sulfonic acid groups, spherical |
| | beads, 4% of crosslinking, |
| phenol | Acros Organics, grade: extra pure >99% (measured |
| | purity by GC: 99.9%) |
| MEPA | 3-mercaptopropionic acid, Sigma-Aldrich, Purity >99% |
| | (measured purity by GC: 99.3%) |
| acetone | VWR, GPR rectapur >99.5% (measured purity by GC: |
| | 100%, water not included) |
| acetophenone | Sigma Aldrich >=99.0% Cas Nr 98-86-2 |
| methanol | Fisher Analytical grade; GC purity 99.99% |

A column reactor was equipped with 150 g of the phenol-wet catalyst (volume of phenol-wet catalyst in the reactor: 210 to 230 ml). The column reactor was heated to 60° C. (catalyst bed temperature during reaction: 63° C.). A mixture of phenol, acetone (3.9 wt.-%) and MEPA (160 ppm with respect to the sum of the masses of phenol and acetone) was prepared and tempered to 60° C. This mixture was pumped into the column reactor with a flow rate of 45 g/h. The column reactor was equipped with a sampling point at the bottom. Using the aperture of the sampling point, different samples were taken during the reaction. Sampling time was 1 h and the amount of the sample taken each hour was 45 g.

A first run (standard run) was conducted for 52 h. After 48 h, 49 h, 50 h and 51 h, respectively, a sample was taken and analyzed via GC.

A second run (impurity run) was conducted for 52 h. At the beginning of the second run 1670 ppm (with respect to the sum of the masses of phenol and acetone) of acetophenone was dosed to the reaction system. After 48 h, 49 h, 50 h and 51 h, respectively, a sample was taken and analyzed via GC. After this a fresh mixture of acetone, phenol and MEPA was used and a third run (standard run) was conducted for 52 h. After 48 h, 49 h, 50 h and 51 h, respectively, a sample was taken via a syringe and analyzed via GC. Then a fourth run (impurity run) was conducted for 52 h. At the beginning of the fourth run 1680 ppm (with respect to the sum of the masses of phenol and acetone) of acetophenone was dosed to the reaction system. After 48 h, 49 h, 50 h and 51 h, respectively, a sample was taken and analyzed via GC. Finally, a fifth run (standard run) was conducted for 52 h. After 48 h, 49 h, 50 h and 51 h, respectively, a sample was taken and analyzed via GC.

The gaschromatography (GC) for methanol was conducted using a column Agilent J&W VF-1MS (100% Dimethylpolysiloxane) of the size 50 m×0.25 mm×0.25 μm, a temperature profile of 60° C. for 0.10 min, heating with 12° C./min to 320° C. and holding this temperature for 10.00 min; injecting 1 μl with a split of 10/1 at 300° C.); wherein the flow is 2 ml/min at an initial pressure of 18.3 psi (1.26 bar)

The gaschromatography (GC) for acetophenone, phenol, para,para BPA were conducted using a column Agilent J&W VF-1MS (100% Dimethylpolysiloxane) of the size 50 m×0.25 mm×0.25 μm, a temperature profile of 80° C. for 0.10 min, heating with 12° C./min to 320° C. and holding this temperature for 10.00 min; injecting 1 μl with a split of 10/1 at 300° C.); wherein the flow is 2 ml/min at an initial pressure of 18.3 psi (1.26 bar)

The standard run represents the reaction of acetone and phenol in the presence of the catalyst and cocatalyst to form BPA. From this the acetone conversion can be estimated including respective error bars. This conversion represented the baseline to evaluate whether the impurities influence the catalyst deactivation or not. The acetone conversion of standard runs 3 and 5 were compared to the value of standard run 1 to determine the effect of acetophenone on the catalyst. If the acetone conversion dropped out of this conversion, it would be proven that acetophenone has an effect on the BPA catalyst. In order to show that this kind of evaluation can be used to determine catalyst poisoning, a reference run was conducted using methanol as impurity. It is known from the state of the art that methanol is a known poison for the catalyst in the BPA process that is described for example in U.S. Pat. No. 8,143,456. Table 1 shows the respectively obtained results. The values given in the table are the average values obtained from the four samples taken during each run (after 48 h, 49 h, 50 h and 51 h).

TABLE 1
reference run with methanol

| | | first run | second run | third run | fourth run | fifth run |
|---|---|---|---|---|---|---|
| | | (standard | (impurity | (standard | (impurity | (standard |
| Substance | Unit | run) | run) | run) | run) | run) |
| Acetone | % | 82.63 | 78.65 | 81.92 | 78.20 | 79.42 |
| conversion | | | | | | |
| Methanol | mg/kg | — | 1710 | — | 1660 | — |
| IN** | | | | | | |

**The amount of methanol IN is measured before the catalyst.

As can be clearly seen from table 1, the acetone conversion of each standard run 1, 3 and 5 drops. This means that the catalyst is poisoned by methanol and the conversion cannot be recovered due to irreversible reactions which decrease the catalyst activity.

The following table shows the results of the first run (standard run), the second run (impurity run), the third run (standard run), the fourth run (impurity run) and the fifth run (standard run) for acetophenone as impurity. The values given in the table are the average values obtained from the four samples taken during each run (after 48 h, 49 h, 50 h and 51 h).

TABLE 2
acetophenone

| | | first run | second run | third run | fourth run | fifth run |
|---|---|---|---|---|---|---|
| | | (standard | (impurity | (standard | (impurity | (standard |
| Substance | Unit | run) | run) | run) | run) | run) |
| Acetone | % | 84.94 | 84.36 | 87.05 | 82.95 | 83.65 |
| conversion | | | | | | |
| Acetophenone | mg/kg | — | 1670 | | 1680 | |
| IN** | | | | | | |
| Acetophenone | mg/kg | — | 1627 | | 1630 | |
| OUT** | | | | | | |

**The amount of acetophenone IN is measured before the catalyst. The amount of acetophenone OUT is measured from the four samples taken during each run (after 48 h, 49 h, 50 h and 51 h; average value).

As can be seen from the results of table 2, the addition of acetophenone in a reaction of phenol and acetone to para,para-BPA leads to almost no drop in acetone conversion for the standard runs 1, 3 and 5. This means that acetophenone is no poison for the catalyst system used. This effect can be seen after each impurity run. Moreover, it can be seen that the acetophenone does not seem to react at all in the system (acetophenone OUT is almost equal to acetophenone IN).

1. A process for preparing ortho,para-, ortho, ortho- and/or para,para-bisphenol A comprising the step of
(a) condensing raw phenol and raw acetone in the presence of a catalyst system, wherein the catalyst system comprises an ion exchange resin catalyst and a sulfur containing cocatalyst,
wherein the amount of acetophenone present in step (a) is higher than 1 ppm with respect to the total weight of the raw phenol.

2. The process according to claim 1, wherein at least part of the sulfur containing cocatalyst is neither covalently nor ionically bound to the ion exchange resin catalyst at the beginning of process step (a).

3. The process according to claim 1, wherein the amount of acetophenone present in step (a) is higher than 1 ppm and equal to or lower than 5000 ppm with respect to the total mass of the raw phenol.

4. The process according to claim 1, wherein the acetophenone is present throughout the whole of process step (a).

5. The process according to claim 1, wherein the process further comprises the following step:
(b) separating the mixture obtained after step (a) into a bisphenol A fraction comprising at least one of ortho,para-, ortho, ortho- or para,para-bisphenol A and a phenol fraction, wherein the phenol fraction comprises unreacted phenol and acetophenone.

6. The process according to claim 5, wherein the separation in step (b) is performed using a crystallization technique.

7. The process according to claim 5, wherein the process further comprises the additional step of
(c) using at least a part of the phenol fraction obtained in step (b) as educt in step (a).

8. The process according to claim 1, wherein the sulfur containing cocatalyst is selected from the group consisting of mercaptopropionic acid, hydrogen sulfide, alkyl sulfides mercaptoalkylpyridines, mercaptoalkylamines, thiazolidines, aminothiophenols and mixtures thereof.

9. The process according to claim 1, wherein the acetophenone present in step (a) is introduced into the process of step (a) as impurity in the raw phenol.

10. A process for preparing polycarbonate comprising the steps of
(i) obtaining a ortho,para-, ortho, ortho- and/or para,para-bisphenol A according to the process of claim 1 and
(ii) polymerizing the ortho,para-, ortho, ortho- and/or para,para-bisphenol A obtained in step (i).

11. The process according to claim 10, wherein the process of step (i) further comprises a step of purifying the ortho,para-, ortho, ortho- and/or para,para-bisphenol A in order to reduce the amount of acetophenone in the ortho,para-, ortho, ortho- and/or para,para-bisphenol A.

12. The process according to claim 11, wherein the step of purifying is performed using a crystallization technique.

13. The process of claim 2, wherein at least 75 mol % of the sulfur containing cocatalyst of the catalyst system is neither covalently nor ionically bound to the ion exchange resin catalyst at the beginning of process step (a).

14. The process of claim 8, wherein the alkyl sulfide is ethyl sulfide.

15. The process of claim 10, wherein step (ii) is performed in the presence of at least one further monomer in order to obtain a polycarbonate.
